(19) United States
(12) Patent Application Publication
(10) Pub. No.: US 2004/0058008 A1
(43) Pub. Date: Mar. 25, 2004

(54) Microparticles having serum as a dispersing agent and process for their preparation and use

(76) Inventors: Peter J. Tarcha, Lake Villa, IL (US); Dale E. Hauenstein, Gurnee, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/251,200

(22) Filed: Sep. 20, 2002

(51) Int. Cl. A61K 35/16; A61K 9/16; A61K 9/50; A61K 31/4745; A61K 31/337

(52) U.S. Cl. 424/490; 424/531; 514/291; 514/449

(57) ABSTRACT

Micron and submicron microparticles prepared with whole, untreated serum as a dispersing agent or surfactant to reduce aggregation and facilitate treatment and administration of localized injury or disease with reduced tendency of adverse immune response. Also disclosed are processes for preparing biodegradable and biocompatible microparticle compositions by emulsifying a solution of polymer dissolved in an organic solvent with a dispersing agent, preferably porcine serum, human serum or a serum autologous to the recipient being treated, in water. The organic solvent is removed, resulting in the formation of solid particles containing active agent. Lyophilized particles can be redispersed using whole, untreated serum according to methods of the invention. The microparticle compositions may be administered to patients for treatment of localized injury or disease.

### FIELD OF THE INVENTION

This invention is directed to methods to produce redispersible, biodegradable microparticles from an aqueous environment using autologous serum, human serum or porcine serum as a dispersing agent and microparticle compositions that are produced by the method. The invention is also directed to the microparticles produced by these methods and methods of treating localized disease or injury using microparticles.

### BACKGROUND OF THE INVENTION

Microparticles are used to deliver an active agent in a controlled manner to animals and humans. Microparticles are effective for such use when their structure is maintained until the microparticle arrives at a desired point of delivery for the active agent and/or when the agent is released from the microparticle at a desired time after administration of the microparticle. Release of the active agent is due to break down or breach of a polymer encapsulating the active agent or by diffusion of that agent. As active agents, microparticles may, for example, contain pharmaceutical, nutritional or biological agents, proteins, chemical compounds or elements, or may be used to deliver nucleic acid sequences.

Parenteral administration of active agents is used to treat localized disease or injury. Localized areas of disease or injury may be particularly difficult to treat or may require large doses of active agent because oral administration or intravenous administration of the active agent increases the time of delivery to the site of disease or injury. During the time between administration and arrival at the site of injury or disease, the active agent may be hydrolyzed, metabolized, or otherwise break down, prematurely inactivating the agent and diluting the dose delivered to the site of disease or injury. In order to deliver therapeutic dosages of active agent at the site where it is needed and eliminate dosing unintended tissues, drugs can be administered locally, contained in a carrier or controlled release matrix such as a biodegradable microparticle. It is also important for precise and accurate delivery of the active agent that the carrier avoids aggregation, i.e., that substantially discrete particles are delivered to the intended site. After delivery, the active agent may be released at a controlled rate at the site of trauma or disease in order to maximize local concentration and efficacy as well as minimize systemic side effects.

Factors that affect the integrity of the microparticle include polymer characteristics such as biodegradability, swellability, permeability, temperature and pH sensitivity. Polyanhydrides are easily hydrolyzed by exposure to moisture and have been generally avoided for use in aqueous environments. In the past, preferred polymers used to encapsulate active agents in microparticles have included polymers other than polyanhydrides. This invention provides improved means for using hydrolytically unstable, biodegradable polymers, such as polyanhydrides, polyorthoesters, and polylactides as controlled release micro- and nanoparticulate delivery systems.

Successful delivery of the active compound to a targeted site also depends upon discrete microparticles being delivered (dispersed) at the site of interest rather than having aggregates or clumps of several microparticles delivered to the targeted site. Otherwise, microparticles toward the center of such aggregates may not deliver the active compound to the target site because breakdown of the polymer at the outer surfaces of the microparticle is inhibited or retarded at critical times.

Generally, microparticles are formed by encapsulating the active agent inside a polymer encasement. In conventional techniques, microparticles intended for administration parenterally are prepared by dissolving a water-insoluble polymer in a solvent along with the active agent. Dispersion of the polymer solution into microdroplets is induced by homogenization into an aqueous phase that contains a surfactant. The aqueous emulsion contains the microdroplets. Solvent is evaporated, resulting in a solid microparticle dispersion. The microparticles are thereafter ready for use, or they may undergo lyophilization for long-term storage.

After the solvent is removed, a surfactant coating forms the outer surface of the microparticle. A variety of surfactants is available commercially for the formation of stable emulsions, but for in vivo human treatments especially, it is undesirable to introduce foreign substances. In fact, many of these, for example, serum components derived from non-human species, are not suitable for in vivo human use. Such agents can even be toxic or induce tissue inflammation or other immune reactions unless they are highly purified or otherwise treated prior to use to minimize adverse reactions. Therefore, these surfactants are produced synthetically or purified forms derived from natural sources are used. For example, U.S. Pat. No. 6,120,805 teaches microparticles prepared using serum albumin, a protein derived from blood serum, as an exemplary surface active agent. International Patent WO 96/20698, assigned to University of Michigan, discloses the use of selected biological components including hemoglobins, myoglobins, albumins, proteins and peptides as surface modifying agents for particles containing drugs.

There remains a need for biodegradable and biocompatible microparticles of small dimensions with reduced aggregation in use (e.g., remain suspended as individual particles at administration) and reduced tendency for compositions containing these microparticles (e.g., suspensions) to induce tissue inflammation or immune reactions. Further, there is need for microparticles of relatively small diameter and appropriate structural stability for the treatment of localized disease or injury, e.g., in coronary tissues or cancer. It would be especially advantageous to use endogenous compositions as surfactants to simplify formation, reconstitution and administration of microparticle compositions to patients needing treatment without increasing the occurrence of adverse reactions. It would also be advantageous to facilitate delivery of microparticles, for example, at localized tissues by injection or via catheter.

The present invention provides these features and advantages and more by providing biocompatible and biodegradable microparticles containing a pharmaceutically or biologically active agent in a polymer within a surfactant coating. The present invention also provides methods for producing such microparticles as well as biodegradable and biocompatible compositions comprising microparticles and a method for administering such compositions to a patient.

### SUMMARY OF THE INVENTION

In one aspect, the present invention provides a microparticle composition having an active agent; a polymer encapsulating the active agent; and a serum, where the serum is selected from the group consisting of whole, untreated serum and a whole, untreated serum in aqueous solution.

The present invention also relates to biodegradable compositions containing microparticles prepared according to the inventive methods dispersed in a serum.

In another aspect, the present invention provides processes for preparing microparticle compositions including the following steps. An aqueous phase containing a serum is prepared, wherein the serum is whole untreated serum or whole, untreated serum in aqueous solution. An organic phase containing a polymer and an active agent is mixed with the aqueous phase to form an immiscible system. The two-phase system is dispersed to form microparticles containing the active agent and the polymer. Processes according to the invention can optionally include a step of redispersing said microparticles in a serum. In preferred embodiments, the serum has a concentration in the two-phase system of at least about 1.0% (v/v).

Porcine serum, human serum and a serum autologous to an intended recipient of the microparticle are preferred for the present invention.

A variety of polymers can be used in the present invention. Preferred polymers are polyanhydrides, more preferred are embodiments where the polyanhydride is poly(1,3-bis(p-carboxyphenoxy)propane-co-sebacic acid having a weight ratio of carboxyphenoxypropane to sebacic acid of between about 1:1 and about 1:8. An especially preferred embodiment is a polyanhydride where the ratio of carboxyphenoxypropane to sebacic acid is about 1:4.

A variety of active agents for human or animal patient administration can be delivered by the microparticles of the present invention. Especially desirable active agents are anti-proliferatives, such as rapamycin, paclitaxel or derivatives thereof, matrix metalloproteinase inhibitors and anti-cancer agents.

Preferred particles according to the present invention have an average particle diameter of less than about 20 microns or preferably of about 1 micron or less.

According to another aspect, the present invention also relates to methods of treating a patient having a localized site of disease or injury. In these inventive methods, a biodegradable composition including microparticles having an active agent, a polymer encapsulating the active agent and a serum associated with at least a portion of the polymer. The serum is selected from the group consisting of whole, untreated serum and whole, untreated serum in aqueous solution. An effective amount of the composition is administered to a patient, for example by injection or via catheter. Methods according to this aspect of the invention are suitably used where the localized site is a vessel, including for example, a lymphatic vessel, within a coronary artery, myocardial tissue or tumor.

### BRIEF DESCRIPTION OF THE DRAWINGS

FIG. 1 illustrates an exemplary disperser set up to circulate fluids during the emulsification process used in methods according to the present invention.

FIG. 2 is a photomicrograph of a dispersion of microparticles prepared with porcine serum as a dispersing agent.

FIG. 3 is a histological section (240× magnification) of an explanted pig heart myocardial tissue containing injected microparticles of the present invention.

FIG. 4 is a histological section (240× magnification) of an explanted pig heart myocardial tissue containing injected buffer as a control.

### DETAILED DESCRIPTION OF THE INVENTION

The present invention will be better understood by reference to the following definitions.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

By “aggregate,” it is meant the reversibly or irreversibly associated particles in suspension or as a precipitate.

By “continuous phase,” it is meant a liquid medium into which particulates or microdroplets are dispersed or suspended.

By “dispersion,” it is meant a suspension of particulates, which may include microdroplets in a continuous phase. As those skilled in the art will appreciate, the term “dispersion” also refers to producing a heterogeneous suspension, emulsion, or colloid by the application of energy to at least two phases that are not miscible with each other. The energy may be applied by stirring, high shear mixing, or ultrasonication. The particular meaning of the term herein will be clear to those skilled in the art by the context in which the term appears.

By “emulsion,” it is meant a suspension of at least one immiscible liquid droplet phase within another liquid phase.

By “microparticle,” it is meant a particulate having a diameter greater than or equal to 1 micron.

By “nanoparticle,” it is meant a particulate having a diameter less that 1 micron.

By “redispersion,” it is meant reconstitution of particulates, formed from a liquid phase, from the dry state to a dispersed state.

By “suspension,” it is meant a two or more phase system wherein one phase is continuous and the second phase (or other phases) is discontinuous but substantially evenly distributed within the continuous phase.

By “surfactant,” it is meant a substance that is soluble in, but localizes at the interface of two phases in a dispersion to avoid or minimize aggregation of one phase, thereby stabilizing the dispersion. Other terms used to describe a surfactant are: surface-active agent and stabilizer.

All references, including publications, patent applications, and patents, cited herein are hereby incorporated by reference to the same extent as if each reference were individually and specifically indicated to be incorporated by reference and were set forth in its entirety herein.

Generally, the present invention is directed to biodegradable and biocompatible microparticles containing active agents and methods of preparing these microparticles from an aqueous solution using porcine serum, human serum or a serum autologous to the animal to be administered the microparticle. Very small microparticles of 1 micron diameter or less can be prepared according to methods of the present invention. According to other aspects of the invention, whole untreated serum may be used in microparticle formation, dispersion, or redispersion or at each of these stages. As a result, microparticle aggregation is reduced and administration of compositions according to the invention is facilitated. In particular, the present invention enables and enhances delivery of substantially unaggregated (i.e., discrete) microparticles, controlled delivery of a desired dosage of a variety of active agents and treatment of localized sites.

In preferred embodiments, the polymer component of the inventive microparticle is a polyanhydride and the invention produces, by the methods disclosed, a microparticle of an average size that is 1 micron or less (e.g., microparticle or nanoparticle dimensions).

Serum or serum diluted in aqueous solution is the continuous phase used in the microparticle preparation, dispersion or redispersion stages described herein. Preferably, porcine serum or a serum that is autologous to the subject receiving the microparticle, which may be man, is used. The serum used may be whole, untreated serum obtained directly, e.g., without separation of endogenous components or purification, from the patient to be treated with a microparticle-containing composition according to the invention. However, it may be desired to modify pH and ionic strength of the serum or serum-in-water solution, or add additives or emulsion stabilizers or coatings to the aqueous solution. Such stabilizers or coatings-may be inorganic salts, bovine serum albumin, dextrose, methylcellulose, or polyvinylpyrrolidone. These additives, stabilizers or coatings are optional however. The serum used may include a preservative, such as sodium azide in a concentration of about 0.03 weight percent, but the concentration may range from about 0 to 1 wt %. Significant reduction in aggregation of the microdroplets or microparticles, depending on the stage of the process, is observed when the serum is at least 0.1 volume percent of the dispersion.

Aggregation of microparticles to one another can inhibit delivery of active agents to the targeted site of treatment. The use of porcine serum in microparticle formation in concentrations of at least about 1.0% by volume prevented aggregation of the microparticles. Porcine serum or a serum autologous to the animal or human to be administered the microparticle are preferred dispersion agents.

Any of a variety of polymers that form aqueous dispersions may be used to prepare the microparticles of the present invention. A suitable polymer may be of any molecular weight as long as it provides sufficient repeat units and is not or does not become too viscous for processing; preferred molecular weights are between 10,000 to 200,000. Exemplary polymers include polyanhydrides, polyesters, polyamines, polyurethanes, polyorthoesters, polyacrylonitriles, poly(lactic acid) and polyphosphazenes or other hydrophobic polymers.

Polyanhydrides are preferred. They are easily hydrolyzed by exposure to aqueous environments and therefore are especially useful in the preparation of microparticles for compositions to be administered directly, or nearly directly, to a localized site of disease or injury. Representative polyanhydrides useful in practicing the present invention include, for example, poly(1,3-bis(p-carboxyphenoxy)propane-co-sebacic acid) with a preferable monomer weight ratio of 1:4 (carboxyphenoxy propane: sebacic acid), but may be in a range of ratios of between about 1:1 and about 1:8. Another suitable polyanhydride is poly(sebacic acid-co-erucic acid dimer), having a weight ratio of sebacic-to-erucic acid dimer of 1:1. Those skilled in the art can discern other suitable weight ratios for this and other polymers used.

Active agents or compounds for encapsulation in the microparticles of the present invention generally include, but are not limited to, pharmaceutical, nutritional, biological agents, therapeutic agents, diagnostic agents and chemical compounds that would be useful for site-specific treatments or procedures. Exemplary active agents that may be administered to a human or animal patient by use of the microparticles of the present invention include biologically active agents such as nucleic acids, growth factors, angiogenesis inhibitors, hormones, proteins, cells, antibodies, antigens, cellular components and viruses or viral components either live or inactivated; chemical elements or compounds; pharmaceutical compounds; magnetic and/or optical markers; dyes (e.g., to enhance visualization in the tissue); and nutritional supplements.

Pharmaceutical compounds such as matrix metalloproteinase inhibitors such as that commercially available as BATIMASTAT compound (British Biotech plc, London, UK), antiproliferatives like farnesyltransferase inhibitor, paclitaxel, or rapamycin and derivatives thereof, to extend the patentcy rates for vascular grafts or stents by inhibiting neointimal hyperplasia are preferred active compounds for preparation of the microparticle of the present invention and treatment of localized injury or disease by the methods described herein. The microparticles of the invention are also preferably used in the controlled delivery of locally administered antibiotics, such as gentamycin or vancomycin, to prevent infection in a vascular graft or stent, to prevent restenosis, or for the controlled delivery of locally administered anti-inflammatory drugs such as, for example, dexamethasone to reduce the infiltration of macrophages with subsequent release of cytokines. Local administration of anti-cancer drugs such as, for example, farnesyltransferease inhibitor or cisplatin, or 5-fluorouracil if that drug is administrated directly to the liver, to inhibit the growth of solid tumors is also preferable. Growth stimulating factors such as vascular endothelial growth factor may also be delivered.

The active compounds are encapsulated in the polymer by the inventive methods, including the steps of forming an immiscible mixture or emulsion and then removing the organic solvent by evaporation. The preferred method of microparticle formation is by subjecting the mixture to shear forces. Such forces are achieved by, for example, dispersing or forcing the mixture through a pneumatic nozzle or microfluidizer processor. The most preferred method is a combination of subjecting the mixture to a high shear disperser to form microdroplets and then further reducing the droplet size by processing the emulsion with a microfluidizer or by ultrasonification, and finally removing the organic solvent by evaporation, resulting in a dispersion of solid micro-or nanoparticles containing the active agent.

Microparticle size depends largely upon the selection of equipment to induce emulsification of the polymer solution in the aqueous phase, the characteristics of the polymer selected for use in the microparticle, and the surfactant or surfactants which inhibit particle aggregation. Particle size is an important characteristic of a dispersion, typically being between 10 and 1000 nm with an upper limit near 50 microns. Polyanhydride microparticles of a particle size of approximately 15-20 microns were produced according to the invention using a high shear disperser, namely the Ultra-Turrax® Disperser, with a maximum shear rate of 24,000 min⁻¹. The aqueous emulsion was subjected to a minimum shear rate of about 11,000 min⁻¹ that was increased to the maximum shear during the processing. Finished polyanhydride particles were analyzed for changes in molecular weight due to processing. Typically, the weight-average molecular weight (M_{w}) and the number average molecular weight (Mₙ) of the polyanhydride particles decreased by 28% and 18%, respectively.

One specific method by which microparticles are prepared uses double emulsion. In one illustrative embodiment of the invention, microspheres containing water-soluble bioactive agents (e.g., peptide or drug) are prepared using a water-in-oil-in-water double emulsion method, which method comprises the steps of: (a) dissolving at least one polymer and a surfactant in a water-immiscible organic solvent to yield an organic phase; (b) dissolving a water soluble bioactive agent in aqueous solution to yield a first aqueous phase; (c) emulsifying the organic and first aqueous phases to yield a first milky emulsion containing water droplets plus active agent dispersed in a continuous organic phase; (d) dissolving a second surfactant in an aqueous solution to yield a second aqueous phase; (e) emulsifying the first milky emulsion and the second aqueous phase to yield a second milky emulsion; and (f) removing the organic solvent from the second milky emulsion to yield microspheres containing the water-soluble bioactive agent. The removal of the organic solvent in the final step is preferably by means of evaporation.

Polyanhydride microparticles may be filtered by standard means, such as through glass wool, synthetic fiber, cellulosic or other natural fibers, between the high shear dispersing process and the microfluidizer or ultrasonification process. During the microfluidization process, the microparticles are preferably subjected to a maximum pressure of about 30,000 psi. Microparticles that were subjected to microfluidization subsequent to the dispersion process were determined to have particle sizes in the range of less than 1 micron. Comparatively, microparticles prepared by cryogenic milling resulted in a particle size distribution of larger than 45 microns for 95.4% of the particles.

Microparticles of the present invention may be lyophilized and redispersed prior to being administered to a subject or patient by methods that include oral ingestion, intravenous administration, or preferably by direct injection or by injection through a catheter. Redispersion of the microparticles is preferably in a solution of serum to avoid aggregation of the microparticles; however, the microparticles can be easily redispersed by the addition of 0.9% saline. The dispersed microparticles may be administered in combination with a pharmaceutically acceptable carrier. A pharmaceutically acceptable carrier is a non-toxic, inert solid, semi-solid or liquid filler, diluent, or auxiliary formulation of any type as known in the art. Examples of pharmaceutical carriers include, for example, sodium citrate, dicalcium phosphate, starches, lactose, sucrose, glucose, mannitol and silicic acid.

The microparticles may be administered to a patient or subject in an effective amount. An effective amount of a microparticle composition prepared by the methods described herein is a sufficient amount of the compound to provide the relief desired at a reasonable benefit/risk ratio applicable to any medical treatment. For treatment in humans, the total dosage of the composition or compounds will be decided by the attending physician within the scope of sound medical judgment. The specific effective amount or effective dose level for any particular patient or individual will depend upon a variety of factors including the injury or disease being treated and severity of injury or disease; the activity of the compound being administered; the specific composition used; the age, body weight, general health, gender and diet of the patient; the timing of administration of the compound relative to other significant or related events; route of administration; rate of elimination of the compound; duration of treatment; drugs or therapy that may be used in combination with the compound being administered and other factors well known in the field of medical arts.

While some potential advantages and objects have been expressly identified herein, it should be understood that some embodiments of the invention may not provide all, or any, of the expressly identified advantages and objects.

The following examples further illustrate the invention but, of course, should not be construed as in any way limiting its scope. Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

Throughout these examples, poly(CPP:SA) is the notation for poly(1,3-bis(p-carboxyphenoxy)propane-co-sebacic acid). Examples 1 and 2 are comparative examples, illustrating the utility of sufficient impact force (shear) to produce small microparticles. The use of a high shear disperser with a maximum shear of 24,000 min.⁻¹ produces microparticle sizes approaching 15 to 20 microns.

### Example 1

### Production of Polyanhydride Microparticles Using the Ultra-Turrax® Disperser

An aqueous suspension of a polyanhydride (poly(CPP:SA) with a 1:4 weight ratio) was prepared using porcine serum as a dispersing agent to produce microparticles on the order of 15 microns and less in diameter. To this end, two separate solutions (an organic phase containing the polymer and an aqueous phase containing the porcine serum) were prepared and mixed together in a high shear disperser.

Solution A—Into a 125-ml flask was added 75 mls of purified water and 10 mls of porcine serum (Sigma, P-9783) containing 0.03 wt % sodium azide (Aldrich, 43,845-6) as a preservative.

Solution B—To a 50-ml flask was added 3.1 grams of poly(CPP:SA) (1:4) (prepared by Abbott Laboratories Specialty Products Division) followed by 10 mls of dichloromethane (Aldrich 27,099-7). The mixture was swirled and then stirred with a spatula at room temperature until all of the polymer was dissolved.

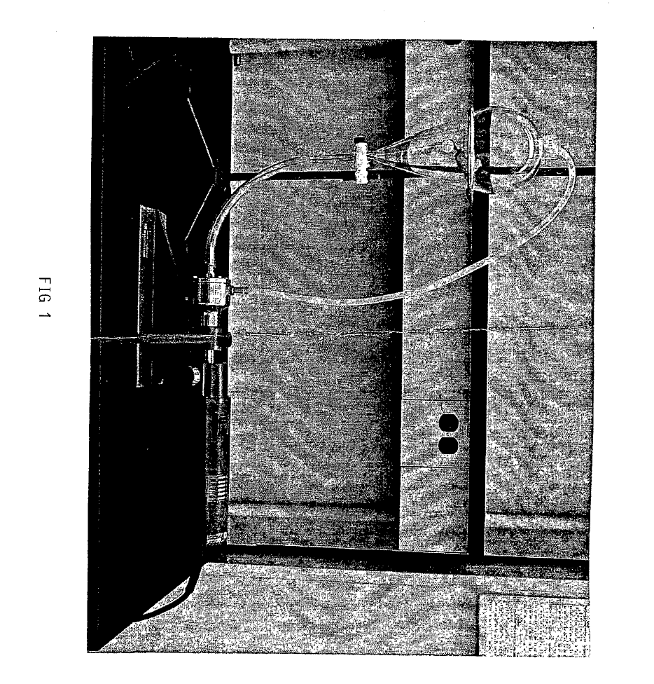

Dispersion—Solution A was poured into the 250-ml separatory funnel of the dispersion set-up as shown in FIG. 1. The stopcock of the separatory funnel was opened and solution A allowed to flow into the dispersion system. The disperser (Ultra-Turrax® UTL 25 basic inline from IKA Works, Inc. of Wilmington, N.C.) was run at low shear (11,000 min⁻¹). The disperser was turned off and solution B was added to the separatory funnel. As soon as solution B was added, the disperser was turned on low shear for 10 seconds, then increased to its highest shear (24,000 min⁻¹) and kept at this rate for 5 minutes. The mixture was recycled into the separatory funnel as shown in FIG. 1. After 5 minutes, the milky solution was collected in a 125-ml flask, capped, and stored in a refrigerator. Microdroplets did settle out after 30 minutes, but easily went back in suspension upon swirling.

Removal of dichloromethane—The dichloromethane was removed by flowing nitrogen gas over the sample for 1 hour at room temperature. To ensure that all dichloromethane was removed, the suspension was heated to 70° C. while flowing nitrogen gas over the sample for an additional 20 minutes.

Particle size distribution was evaluated by placing two drops of the dispersion on a microscope slide, covered with a microscope slide cover, and using a Leitz Ortholux II microscope at a magnification of 125×. Droplet size varied up to approximately 15 microns.

Microparticle wash—Two 50-ml glass centrifuge tubes were filled with equal volumes of milky suspension and placed in a Jouan C412 centrifuge at 2500 rpm for 5 minutes. After 5 minutes, the supernatant from each tube was removed. Equal amounts (approximately 25 mls) of purified water were added to each tube and the solid in each tube dispersed with a glass pipette, swirled and placed back into the centrifuge at 2500 rpm for 5 minutes. After 5 minutes, the supernatant from each tube was removed, fresh purified water added (approximately 25 mls), solid dispersed, and then placed back into the centrifuge at 2500 rpm for 5 minutes. This constituted 2 washings with purified water. After 5 minutes, the supernatant from each of the two tubes was removed. A small portion of the solid was placed in 1 ml of 0.01 M phosphate buffered saline, vortexed and sonicated for 1 minute. The particle size distribution as evaluated on the Leitz Ortholux II microscope (magnification of 125×) varied up to approximately 15 microns as previously observed. To each of the two tubes containing solids was added approximately 15 mls of fresh purified water. Each sample was dispersed with a glass pipette, swirled and poured into a 500-ml Labconco glass lyophilization vessel. The tubes were rinsed several times with fresh purified water and poured into the glass vessel.

Lyophilization—The Labconco glass lyophilization vessel containing the washed microparticle sample was chilled in a dry ice-acetone bath and attached to a Labconco Lymph-Lock 6 Freeze Dry/Shell Freeze System with a vacuum of 7×10-3 Mbar and collector temperature of −47° C. Freeze-drying was allowed to proceed overnight (approximately 20 hours). The dry microparticles were collected, placed in a plastic bag, and stored at −80° C.

### Example 2

### Production of Polyanhydride Microparticles Using a Higher Polymer Concentration and the Ultra-Turrax® Disperser

As in Example 1, an aqueous suspension of a polyanhydride (poly(CPP:SA) with a 1:4 weight ratio) was prepared using porcine serum as a dispersing agent to produce microparticles on the order of 15 microns and less in diameter. To this end, two separate solutions (an organic phase containing the polymer and an aqueous phase containing the porcine serum) were prepared and mixed together in a Ultra-Turrax® high shear disperser.

Solution A—Into a 125-ml flask was added 75 mls of 0.01 M phosphate buffered saline (Sigma, P-4417) of pH 7.4 at 25° C. and 10 mls of porcine serum (Sigma, P-9783) containing 0.03 wt % sodium azide (Aldrich, 43,845-6) as a preservative.

Solution B—To a 100-ml beaker was added 4.1 grams of poly(CPP:SA) (1:4) (prepared in-house) followed by 10 mls of dichloromethane (Aldrich 27,099-7). The mixture was swirled and then stirred with a spatula at room temperature until all of the polymer was dissolved.

Dispersion—Solution A was poured into the closed separatory funnel attached to the dispersion apparatus of Example 1. The stopcock on the separatory funnel was opened and solution was allowed to flow into the dispersion system. The disperser was run at low shear (11,000 min.⁻¹) for about 10 seconds. After 10 seconds, the disperser was turned off and solution B was added to the separatory funnel. As soon as solution B was added, the disperser was turned on at low shear for approximately 10 seconds, then increased to its highest shear (24,000 min.⁻¹) and kept at this rate for 5 minutes. The mixture was recycled back into the separatory funnel during the dispersion process as shown in Example 1. The milky solution was collected in a 125-ml flask.

Removal of Dichloromethane—To remove excess dichloromethane, the milky solution in the 125-ml flask was heated to 40° C. with constant stirring and a nitrogen blanket was allowed to flow over the surface of the liquid for 30 minutes.

Microparticle wash—The suspension was washed with distilled water with the aid of a centrifuge. Two 50-ml glass centrifuge tubes were filled with equal volumes of milky suspension and placed in a Jouan C412 centrifuge at 2500 rpm for 5 minutes. After 5 minutes, the supernatant from each tube was removed. Equal amounts (approximately 25 mls) of purified water were added to each tube and the solid in each tube dispersed with a glass pipette swirled and placed back into the centrifuge at 2500 rpm for 5 minutes. After 5 minutes, the supernatant from each tube was removed, fresh purified water added (approximately 25 mls), solid dispersed, and then placed back into the centrifuge at 2500 rpm for 5 minutes. This constituted 2 washings with purified water. After 5 minutes, the supernatant from each of the two tubes was removed. To each of the two tubes containing solids was added approximately 15 mls of fresh purified water. Each sample was dispersed with a glass pipette, swirled and poured into a 1200-ml Labconco glass lyophilization vessel. The tubes were rinsed several times with fresh purified water and poured into the glass vessel.

Lyophilization—The Labconco glass lyophilization vessel containing the washed microparticle sample was chilled in a dry ice-acetone bath and attached to a Labconco Lymph-Lock 6 Freeze Dry/Shell Freeze System with a vacuum of 7×10-3 Mbar and collector temperature of 47° C. Freeze-drying was allowed to proceed overnight (approximately 20 hours). The dry microparticles were collected, placed in a plastic bag, and stored at −80° C.

Molecular weight determination—The molecular weight distribution was determined using a Waters 2690 Separation Module equipped with a Waters 410 Differential Refractometer in methylene chloride using polystyrene standards. The weight-average and number-average molecular weights of the lyophilized microparticles were 27,000 and 12,000, respectively. These values were 28% and 18% lower, respectively, than the original starting polymer (prior to using this process). The weight-average and number-average molecular weights of the starting polymer were 38,000 and 14,000, respectively.

Microparticle redispersion in porcine serum—Into a 1.5-ml flextube was added 23 mg of lyophilized microparticles, followed by 1 ml of porcine serum (Sigma, P-9783) containing 0.03 wt % sodium azide (Aldrich, 43,845-6) as a preservative. The mixture was vortexed for 10 seconds, then sonicated in an ultrasonic bath (L&R 2014 Ultrasonic Cleaning System) for 2 minutes. Two drops of the dispersion were placed on a microscope slide, covered with a microscope slide cover, and evaluated using a Leitz Ortholux II microscope at a magnification of 125× (FIG. 1) as well as 625×. The dispersion showed no significant microparticle aggregation with particle sizes varying up to approximately 15 micron in diameter.

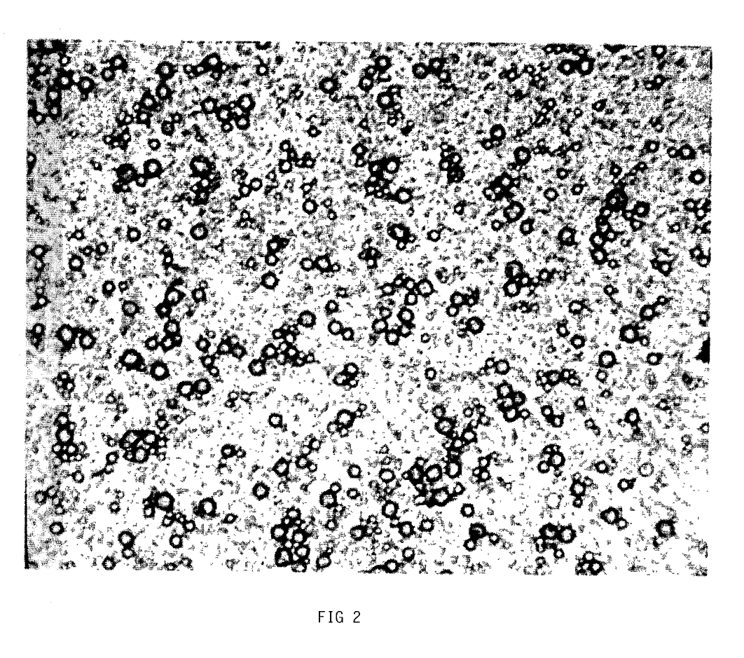

FIG. 2 generally illustrates the effects of the use of porcine serum as a dispersing agent in minimizing aggregation of microparticles. FIG. 2 is a photomicrograph of microparticles that were prepared according to Example 2. The microparticles did not exhibit significant aggregation in the serum.

### Example 3

### Production of Polyanhydride Microparticles Containing Coumarin-6 as a Fluorescent Marker Using the Ultra-Turrax® Disperser

Example 3 shows the incorporation of a fluorescent marker in the process defined in Examples 1 and 2. As in the previous examples, an aqueous suspension of a polyanhydride (poly(CPP:SA) with a 1:4 weight ratio) was prepared using porcine serum as a dispersing agent to produce microparticles on the order of 20 microns and less in diameter. Coumarin-6 was added as a fluorescent marker in order to locate the microparticles upon delivery in tissue. To this end, two separate solutions (an organic phase containing the polymer with the fluorescent marker and an aqueous phase containing the porcine serum) were prepared and mixed together in a high shear disperser.

Solution A—Into a 125-ml flask was added 75 mls of purified water and 10 mls of porcine serum (Sigma, P-9783) without preservative.

Solution B—To a 50-ml beaker was added 5.0 grams of poly(CPP:SA) (1:4) followed by 0.6 milligrams of coumarin-6 (Aldrich 44,263-1). To this combination was added 20 mls of dichloromethane (Aldrich 27,099-7). The mixture was swirled and then stirred with a spatula at room temperature until all of the contents was dissolved.

Dispersion—Solution A was poured into the closed separatory funnel attached to the dispersion apparatus as illustrated in Example 1. The stopcock on the separatory funnel was opened and solution was allowed to flow into the dispersion system. The disperser was run at low shear (11,000 min.⁻¹) for about 10 seconds. After 10 seconds, the disperser was turned off, and solution B was added to the separatory funnel. As soon as solution B was added, the disperser was turned on at low shear for approximately 10 seconds, then increased to its highest shear (24,000 min.⁻¹) and kept at this rate for 5 minutes. The mixture was recycled back into the separatory funnel during the dispersion process as shown in Example 1.

Removal of dichloromethane—The dichloromethane was removed by flowing nitrogen gas over the sample. To this end, a vacuum distillation adapter with a plastic tube (attached to the nitrogen gas line) running through it, was attached to the 500-ml 24/40 fitted flask containing the sample. A stirring bar was added for additional agitation. Nitrogen gas was allowed to flow above the surface of the sample as the sample was stirred for approximately one and one-half hours.

Lyophilization—The Labconco glass lyophilization vessel containing the washed microparticle sample was chilled in a −80° C. freezer with periodic changing of position to ensure uniform freezing. Once adequately frozen, the vessel was attached to a Labconco Freeze Dry System\Lymph Lock 4.5 and allowed to proceed overnight (approximately 15 hours). The vacuum was 45×10⁻³ Mbar with a temperature of 43° C. The dry microparticles were collected, placed in a plastic bag, and stored at −80° C.

Redispersion of microparticles in porcine serum—Into a 1.5-ml flextube was added 13.1 mg of lyophilized microparticles, followed by 1 ml of porcine serum (Sigma, P-9783) containing 0.03 wt % sodium azide (Aldrich, 43,845-6) as a preservative. The mixture was vortexed for 10 seconds, then sonicated in an ultrasonic bath (L&R 2014 Ultrasonic Cleaning System) for 2 minutes. Two drops of the dispersion were placed on a microscope slide, covered with a microscope slide cover, and evaluated using a Leitz Ortholux II microscope at a magnification of 125×. Particle sizes varied up to approximately 20 micron in diameter.

Examples 4 and 5 illustrate the process of the current invention, producing microparticles on the order of 1 micron and less.

### Example 4

### Production of Polyanhydride Microparticles Containing Coumarin-6 as a Fluorescent Marker Using the Ultra-Turrax® Disperser Followed by the use of the Microfluidizer

An aqueous suspension of a polyanhydride (poly(CPP:SA) with a 1:4 weight ratio was prepared using porcine serum as a dispersing agent to produce microparticles on the order of 15 microns and less in diameter. Coumarin-6 was added as a fluorescent marker in order to locate the microparticles upon delivery in tissue. This dye can serve as a model compound for an oil-soluble drug as well. To this end, two separate solutions (an organic phase containing the polymer with the fluorescent marker and an aqueous phase containing the porcine serum) were prepared and mixed together in a high shear disperser followed by processing using a microfluidizer. A preservative, sodium azide, was added to the porcine serum. A typical preparation of the solutions is described below.

Solution A—Into a 125-ml flask was added 75 mls of 0.01 M phosphate buffered saline (Sigma, P4417) where pH was adjusted to 8.62 at 25° C. using 0.01 N sodium hydroxide (J. T. Baker, 5636-O₂) and 10 mls of porcine serum (Sigma, P-9783) containing 0.03 wt % sodium azide (Aldrich, 43,845-6) as a preservative. The pH of the resulting solution was 7.74, and was adjusted back to 8.62 using 30 drops of the 0.1 N sodium hydroxide.

Solution B—To a 50-ml beaker was added 5.0 grams of poly(CPP:SA) (1:4) followed by 1.4 milligrams of coumarin-6. To this combination was added 20 mls of dichloromethane. The mixture was swirled and then stirred with a spatula at room temperature until all of the contents was dissolved.

Dispersion—Solution A was poured into the closed separatory funnel attached to the dispersion apparatus as illustrated in Example 1. The stopcock on the separatory funnel was opened and solution was allowed to flow into the dispersion system. The disperser was run at low shear (11,000 min.⁻¹) for about 10 seconds. After 10 seconds, the disperser was turned off, and solution B was added to the separatory funnel. As soon as solution B was added, the disperser was turned on at low shear for approximately 10 seconds, then increased to its highest shear (24,000 min.⁻¹) and kept at this rate for 5 minutes. The mixture was recycled back into the separatory funnel during the dispersion process as shown in Example 1. The milky, greenish solution was collected in a 250-ml flask.

The solution from the Dispersion process was filtered through glass wool in a glass long stem funnel.

Microfluidizer process—A microfluidizer (Microfluidics Corporation, Model 110Y, Serial Number 96077, equipped with a 30,000-psi maximum interaction chamber which is F20Y, ceramic, and 75 micron) was placed in a container of ice, covering all components, to cool and limit degradation due to heat. Approximately 200 mls of filtered sample from the dispersion process was allowed to pass through the microfluidizer for 6 complete volumes. During the process, each complete volume was collected and poured back into microfluidizer. After 6 complete volumes, the sample was collected and placed in a 500-ml 24/40 fitted flask.

Removal of dichloromethane—The dichloromethane used in this process was removed by flowing nitrogen gas over the sample. To this end, a vacuum distillation adapter with a plastic tube (attached to the nitrogen gas) running through it, was attached to the 500-ml 24/40 fitted flask containing the sample. A stirring bar was added for additional agitation. Nitrogen gas was flowed above the surface of the sample as the sample was stirred for approximately one and one-half hours.

Microparticle wash—Upon removal of the dichloromethane, the dispersion was poured into four 50-ml Kimax centrifuge tubes (with screw caps) at equal volumes, and centrifuged in a Jouan C412 Centrifuge at 2500 rpm for 5 minutes and then increased to 3400 rpm for 3 minutes. After 3 minutes, the supernatant from each tube was removed. Equal amounts (approximately 30 mls) of purified water were added to each tube and the solid in each tube dispersed with a glass pipette swirled and placed back into the centrifuge at 3400 rpm for 5 minutes. After 5 minutes, the supernatant from each tube was removed. The solid microparticles were redispersed in purified water and poured into a 600-ml Labconco glass vessel for freeze-drying.

Particle size analysis prior to lyophilization—Particle size analysis was performed on a sample taken prior to the microparticle wash, a sample of supernatant from the wash, and a sample of washed microparticles. The particle size analysis was performed on a Mastersizer particle analyzer equipped with a small volume sample dispersion unit (Malvern Instruments). All samples were found to have average particle sizes less than 1 micron.

Lyophilization—The washed microparticle sample was placed in a 600-ml Labconco lyophilization vessel, chilled in a dry ice-acetone bath, and attached to a Labconco Lymph-Lock 6 Freeze Dry/Shell Freeze System with an initial vacuum of 244×10⁻³ Mbar and collector temperature of −43° C. Lyophilization was allowed to proceed overnight. The dry microparticles were collected, placed in a plastic bag, and stored at −80° C.

### Example 5

### Production of Polyanhydride Microparticles Containing Coumarin-6 as a Fluorescent Marker Using the Ultra-Turrax® Disperser Followed by the use of the Microfluidizer

As in Example 4, an aqueous suspension of a polyanhydride (poly(CPP:SA) with a 1:4 weight ratio) was prepared using porcine serum as a dispersing agent to produce microparticles on the order of 15 microns and less in diameter. Coumarin-6 was added as a fluorescent marker in order to locate the microparticles upon delivery in tissue. To this end, two separate solutions (an organic phase containing the polymer with the fluorescent marker and an aqueous phase containing the porcine serum) were prepared and mixed together in a high shear disperser, followed by processing in a microfluidizer. Unlike Example 4, no preservative was added with the porcine serum, but this did not alter the final particle characteristics or properties.

### Example 6

### Microparticles Containing the Antiproliferative Agent Rapamycin

Solution A—75 mls of 0.01 M phosphate buffered saline is added into a 125-ml flask along with 10 mls of human serum and the solution is adjusted to a pH of 8.6 using 0.01 N sodium hydroxide.

Solution B—5.0 grams of poly(CPP:SA) (1:4) is added to a 50-ml beaker followed by 1.0 gram of rapamycin. To this combination is added 20 mls of dichloromethane. The mixture is stirred with a spatula at room temperature until all of the contents dissolve.

Dispersion—Solution A is poured into the closed separatory funnel attached to the dispersion apparatus as illustrated in FIG. 1. The stopcock on the separatory funnel is opened and solution is allowed to flow into the dispersion system. The disperser is run at low shear (11,000 min.⁻¹) for about 10 seconds. After 10 seconds, the disperser is turned off and solution B is added to the separatory funnel. As soon as solution B is added, the disperser is turned on at low shear for approximately 10 seconds, then increased to its highest shear (24,000 min.⁻¹) and kept at this rate for 5 minutes. The mixture is recycled back into the separatory funnel during the dispersion process. The emulsion from is then filtered through glass wool in a glass long stem funnel and added to the microfluidizer.

Microfluidizer process—The microfluidizer (Microfluidics Corporation, Model 110Y, Serial Number 96077, equipped with a 30,000-psi maximum interaction chamber which is F20Y, ceramic, and 75 micron) is placed in a container of ice, covering all components, to limit degradation due to heat. Approximately 200 mls of filtered sample from the dispersion process is allowed to pass through the microfluidizer for 6 complete volumes by collecting and pouring back into the microfluidizer as it is running. After the 6^{th} cycle, the sample is collected and placed in a 500-ml 24/40 fitted flask.

Removal of dichloromethane—The dichloromethane is removed by flowing nitrogen gas over the sample. To this end, a vacuum distillation adapter with a plastic tube (attached to the nitrogen gas) running through it, is attached to the 500-ml 24/40 fitted flask containing the sample. A stirring bar is added for additional agitation. Nitrogen gas is allowed to flow above the surface of the sample during stirring for approximately 1.5 hours.

Microparticle wash—Upon removal of the dichloromethane, the dispersion is poured into four 50-ml Kimax centrifuge tubes (with screw caps) at equal volumes, and centrifuged in a Jouan C412 Centrifuge at 2500 rpm for 5 minutes and then increased to 3400 rpm for 3 minutes. After 3 minutes, the supernatant from each tube is removed. Equal amounts (approximately 30 mls) of purified water are added to each tube and the solid in each tube dispersed with a glass-pipette swirled and placed back into the centrifuge at 3400 rpm for 5 minutes. After 5 minutes, the supernatant from each tube is removed. The solid microparticles are redispersed in purified water and poured into a 600-ml Labconco glass vessel for freeze-drying.

Lyophilization—The washed microparticle sample is placed in a 600-ml Labconco lyophilization vessel, chilled in a dry ice-acetone bath, and attached to a Labconco Lymph-Lock 6 Freeze Dry/Shell Freeze System. Lyophilization is allowed to proceed overnight. The dry microparticles are collected, placed in a plastic bag, and stored at −80° C.

### Example 7

### Microparticles Containing a Water-Soluble Bioactive Peptide

Solution A—75 mls of 0.01 M phosphate buffered saline is added into a 125-ml flask along with 10 mls of human serum and the solution is adjusted to a pH of 8.6 using 0.01 N sodium hydroxide.

Solution B—5 mg of VAL-GLY-VAL-ALA-PRO-GLY, the repeating peptide in elastin, is dissolved in 1 ml of distilled water.

Solution C—5.0 grams of poly(CPP:SA) (1:4) is added to a 50-ml beaker followed by 1.0 gram of sorbitol tristearate. To this combination is added 20 mls of dichloromethane. The mixture is swirled and then stirred with a spatula at room temperature until all of the contents dissolve.

Solution B is combined with Solution C on a high shear mixer to produce a water-in-oil emulsion, where the bioactive peptide is contained in the aqueous droplets dispesed in the methylene chloride-polymer phase. This water-in-oil emulsion is added to Solution A and emulsified with a high shear mixer producing a water-in-oil-in water double emulsion

Removal of dichloromethane—The dichloromethane is removed by flowing nitrogen gas over the sample. To this end, a vacuum distillation adapter with a plastic tube (attached to the nitrogen gas) running through it, is attached to the 500-ml 24/40 fitted flask containing the sample. A stirring bar is added for additional agitation. Nitrogen gas is flowed above the surface of the double emulsion during stirring until no methylene chloride vapors are detectable.

Microparticle wash—Upon removal of the dichloromethane, the dispersion is poured into four 50-ml Kimax centrifuge tubes (with screw caps) at equal volumes, and centrifuged in a Jouan C412 Centrifuge at 2500 rpm for 5 minutes and then increased to 3400 rpm for 3 minutes. After 3 minutes, the supernatant from each tube is removed. Equal amounts (approximately 30 mls) of purified water are added to each tube and the solid in each tube dispersed with a glass pipette swirled and placed back into the centrifuge at 3400 rpm for 5 minutes. After 5 minutes, the supernatant from each tube is removed. The solid microparticles are redispersed in purified water and poured into a 600-ml Labconco glass vessel for freeze-drying.

Lyophilization—The washed microparticle sample is placed in a 600-ml Labconco lyophilization vessel, chilled in a dry ice-acetone bath, and attached to a Labconco Lymph-Lock 6 Freeze Dry/Shell Freeze System. Lyophilization is allowed to proceed overnight. Whereby the water trapped within the particles as well as the water making up the continuous phase of the suspension is removed. The dry microparticles containing the bioactive peptide are collected, placed in a plastic bag, and stored at −80° C.

Examples 8 and 9 are comparative, showing that a method such as cryogenic milling does not achieve the necessary particle size distribution.

### Example 8

### Production of Polyanhydride Microparticles by Cryogenic Milling (a Comparative)

Cryogenic milling was performed on a polyanhydride (poly(CPP:SA) with a 1:8 reaction weight ratio) to produce microparticles. To this end, 4.01 grams of the polyanhydride was added to a metal cell containing a magnetic rod and a plug in one end. The cell was closed using a similar plug at the other end. The cell and contents were placed in position in a SPEX Certiprep 6800 Freezer/Mill containing liquid nitrogen and milled. Three milling sessions were performed per run which consisted of an initial 10 minute cooling period, a 2 minute milling session, followed by a 2 minute cooling period between milling sessions. Two consecutive runs were completed on this polyanhydride sample. Upon completion, the sample was taken from the metal cell, placed into a plastic container, and stored at −80° C.

Particle size separation was performed by sieving the cryogenic milled polyanhydride through a 45 micron sieve (U.S. Standard Sieve Series, Sieve Number 325), resulting in a significant majority of particles with size greater than 45 micron (0.173 grams or 4.6% of sample with particle size less than 45 micron and 3.562 grams or 95.4% greater than 45 micron). The particle sizes produced from cryogenic milling are considerably larger than those produced from the process described in Examples 1-5.

### Example 9

### Production of Polyanhydride Microparticles Containing Coumarin-6 as a Fluorescent Marker by Cryogenic Milling

Cryogenic milling was performed on a polyanhydride (poly(CPP:SA) with a 1:4 weight ratio) containing a fluorescent marker (coumarin-6) to produce microparticles. Coumarin-6 was added as a fluorescent marker in order to locate the microparticles upon delivery in tissue. To this end, a concentrate of coumarin-6 in the polyanhydride was produced and this concentrate compounded into fresh polyanhydride to adjust the concentration of the fluorescent marker. Cryogenic milling was then performed on the compounded product.

Coumarin-6/poly(CPP:SA) concentrate—Into a 50 ml crucible was added 2.0063 grams of poly(CPP:SA) with a 1:4 weight ratio (prepared in-house) and 7 mls of a 1.4×10⁻⁴ Molar solution of coumarin-6 in dichloromethane (50.00 mg coumarin-6 to 100 mls with dichloromethane). The mixture was stirred using a spatula and heated in a Fisher Isotemp® 500 Series oven at 88° C. for approximately 15 minutes with occasional stirring. The mixture was cooled to room temperature and stored in a freezer (-24° C.).

Compounding concentrate into poly(CPP:SA)—A mixture composed of 0.039 gram of coumarin-6/poly(CPP:SA) concentrate (from above) and 3.962 grams of fresh poly(CPP:SA) with a 1:4 weight ratio was compounded in a microcompounder (DACA Instruments, Goleta, Calif.) at a temperature of 80° C. and screw speed of 100 rpm. After 5 minutes, the compounded material was extruded, placed in a plastic bag, and stored at −80° C. Two additional compounding runs were completed in the same fashion, extruding after a total time of 6 minutes. One run consisted of 0.038 grams of coumarin-6/poly(CPP:SA concentrate and 3.963 grams of fresh poly(CPP:SA), and the other run contained 0.040 mg and 3.961 grams, respectively. All extrudates were combined and stored at −80° C.

Cryogenic Milling—Cryogenic milling was performed on the compounded material (above) to produce polyanhydride microparticles containing the fluorescent marker. The same procedure as described in Example 6 was followed. To this end, 4.021 grams of the compounded material was added to a metal cell containing a magnetic rod and a plug in one end. The cell was closed using a similar plug at the other end. The cell and contents were placed in-position in a SPEX Certiprep 6800 Freezer/Mill containing liquid nitrogen and milled. Three milling sessions were performed per run which consisted of an initial 10 minute cooling period, a 2 minute milling session, followed by a 2 minute cooling period between milling sessions. A total of 2 consecutive runs were completed on this polyanhydride sample. Upon completion, the sample was taken from the metal cell, placed into a plastic container, and stored at −80° C.

Particle size by microscopy—Particle size distribution was evaluated by optical microscopy. Into a 1½ ml flextube was added 10.52 mg of the cryogenic milled microparticles from above. To this was added 1 ml of a 5% dextrose solution containing 1% porcine serum. The porcine serum contained 0.03 wt % sodium azide as a preservative. The contents of the flextube were vortexed for 5 seconds on a vortexer apparatus (Scientific Industries, Inc., Bohemia, N.Y.) and then sonicated in an ultrasonic bath (L&R 2014 Ultrasonic Cleaning System) for 2 minutes. Three drops of this dispersion was placed on a microscope slide, covered with a microscope slide cover, and evaluated using a Leitz Ortholux II microscope at a magnification of 125×. The majority of particle sizes were observed in the range from approximately 10 to 50 microns. Similarly, the particle size distribution was evaluated on a Nikon, Optiphot 2-Pol, polarizing light microscope using a magnification of 100 and found to again to be between 10 to 50 microns, with larger particles approaching 100 microns in size. A 10.85 mg sample of microparticles in 1 ml of porcine serum (containing 0.03 wt % sodium azide as a preservative) produced similar results. The particle sizes produced from cryogenic milling are considerably larger than observed using the process described in Example 4 and Example 5.

Examples 10 through 15 illustrate the use of porcine serum as a dispersing agent for previously prepared microparticles where polyanhydride microparticles produced by cryogenic milling followed by sieving are used as an example. These particles are not to be confused with those prepared in Examples 1-5, where serum was used as the surfactant in the emulsification process. Examples 10-15 illustrate the utility of porcine serum or, in general, autologous serum to provide well-dispersed microparticles with no significant aggregation and a low potential for causing inflammation. Table 1 summarizes the results.

### Examples 10-15

### Dispersion of Polyanhydride Microparticles in Various Media

The dispersion characteristics of poly(CPP:SA) microparticles (as produced from a sieving operation—not from the process described in the above examples) in various media was evaluated by optical microscopy and compared. In each case, a quantity of poly(CPP:SA), with a 1:4 weight ratio and particle size distribution of aggregate microparticles between 38 micron and 53 micron was placed into a 1½ ml flextube, followed by 1 ml of a particular suspension medium. The quantity of polymer and the identity of the medium are shown in Table 1. The contents of the flextube was vortexed for 5 seconds and then sonicated in an ultrasonic bath (L&R 2014 Ultrasonic Cleaning System) for 2 minutes. Three to four drops of this dispersion were placed on a microscope slide, covered with a microscope slide cover, and evaluated using a Leitz Ortholux II microscope at a magnification of 125×.

TABLE 1
Comparison of dispersion characteristics

| Exam- | Poly (CPP: | Suspension | Dispersion |
|---|---|---|---|
| ple | SA) in mg | Medium | Characteristics |
| 10 | 10.4 | Porcine serum with + | No significant |
| | | 0.03 wt % sodium azide | aggregation |
| | 10.5 | 5% dextrose solution | Significant aggregation |
| 11 | 10.5 | Porcine serum + | No significant |
| | | 0.03 wt % sodium azide | aggregation |
| | 10.14 | 5% dextrose solution | Significant aggregation |
| | 10.09 | 50:50 by volume of por- | No significant |
| | | cine serum/5% dextrose | aggregation |
| | | solution | |
| | 10.25 | 5% dextrose solution | No significant |
| | | containing 1% porcine | aggregation |
| | | serum by volume | |
| 12 | 10.00 | Porcine serum without | No significant |
| | | preservative | aggregation |
| 13 | 10.43 | 5% dextrose solution | Significant aggregation |
| | | containing + 0.5 wt % | |
| | | sodium dodecyl sulfate | |
| | 10.49 | 5% dextrose solution | Moderate aggregation |
| | | containing + 9.1 wt % | |
| | | sodium dodecyl sulfate | |
| 14 | 10.15 | 5% dextrose solution | Moderate aggregation |
| | | containing 1 wt % | |
| | | sodium dodecyl sulfate | |
| 15 | 10.10 | Lactated Ringer's | Significant aggregation |
| | | solution | |

As discussed above, “no significant microparticle aggregation” represents aggregated microparticle sizes typically on the order of or less than the upper limit of 53 microns; “moderate amount of microparticle aggregation” refers to aggregate sizes approaching 100 microns and “significant microparticle aggregation” represents aggregates exceeding 100 microns in size.

There is significant aggregation of the microparticles when an aqueous solution such as a 5% dextrose solution is used as the substrate. The aggregates typically exceed 100 microns in size. However, with the use of porcine serum as the dispersing medium, or dispersing medium compositions containing porcine serum, no significant microparticle aggregates were observed.

Numerous medical treatments may be accomplished using microparticles and methods of the present invention. As those skilled in the art will appreciate, the present invention is especially suitable for delivery instruments (e.g., syringes, catheters) having relatively small diameters. For example, intramyocardial delivery is the most effective way to deliver sensible local concentrations of angiogenic factors, genes and drugs to heart muscle, while minimizing systemic effects. As shown in the following example, freeze-dried biodegradable particles containing drugs can be easily administered and dispersed into myocardial tissue using a helical injection catheter.

### Example 16

### Injection of Polyanhydride Particles into the Myocardium of an Explanted Pig Heart

Polyanhydride microspheres (0.1 g) prepared in accordance with Example 4 were dispersed into 40 ml of 10% porcine serum in phosphate buffered saline, pH—7.4. Biodegradable polyanhydride microspheres (1 micron) were produced by an oil-in-water emulsion technique, using polymer dissolved in methylene chloride as the oil phase and dilute blood serum as the aqueous phase. The serum proteins, which were from the test subject species, served as the emulsion-stabilizing agent, avoiding the need for synthetic colloidal stabilizers that have the potential to induce inflammation.

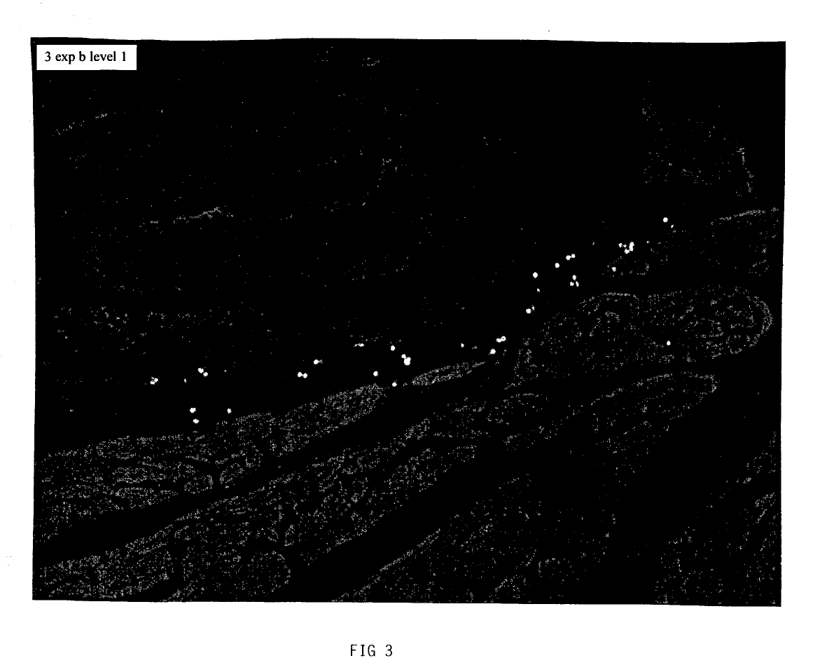

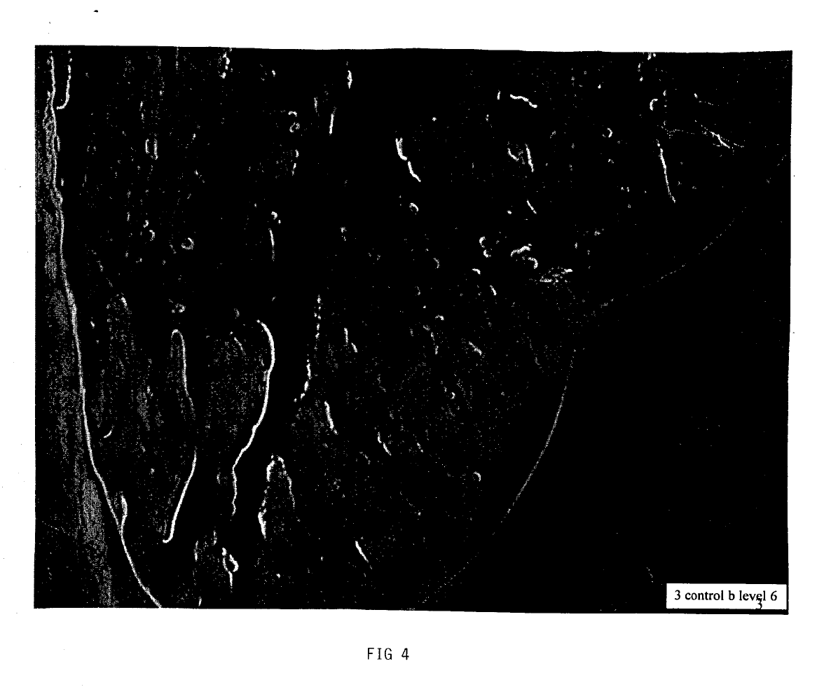

A 1-ml syringe was filled with this suspension and the placed on a Harvard Infusion Apparatus. A Helical Injection Catheter (HIC), manufactured by Biocardia, Inc. (South San Francisco, Calif.) was attached to the output of the infusion apparatus and routed through a BioCardia Universal Guide Catheter to simulate clinical use. The distal (helical) end of the Helical Injection Catheter was guided to a location in the cut-open left ventricle of a pig heart from the endocardial surface. The heart had been opened from the aortic valve down to the apex along the anterior surface, where the septum meets the anterior free wall. The handle of the Helical Injection Catheter was rotated to embed the helix tip in the myocardium 3-5 mm to simulate in vivo use. A volume of 0.1 ml of injectate was infused at a controlled rate at a given site. After the injection was complete, the helix was removed from the tissue by rotating the HIC handle counterclockwise and the catheter tip was repositioned for the next injection. As a control, the process was repeated using buffer instead of the particle suspension. Histological sections of the myocardium containing the injected particles and the buffer control are shown in FIGS. 3 and 4, respectively.

As shown in FIG. 3, under fluorescence microscopy, a good dispersion of discrete spherical particles was seen in histological slides taken around the injection site. The buffer-injected controls exhibited diffuse endogenous fluorescent structures, as shown in FIG. 4. Thus, as those skilled in the art will appreciate, the techniques of the present invention enable the development of injectable sustained release therapy for myocardium (and other) tissue appropriate for safe and routine administration in an interventional setting. Microsphere drug carriers in a broad size range can be administered to localized tissues or sites. In addition, particulate drug carriers can be effectively taken up in the desired tissues, enabling a long range, endogenous drug delivery network (e.g., using the cardiac lymphatic system to treat coronary and myocardial conditions).

The present invention also offers an attractive alternative route for sustained delivery of agents to localized sites such as coronary arteries via the lymphatics draining the myocardium, for example, to prevent restenosis after stent implantation.

What is claimed is:

1. A microparticle composition, comprising:
an active agent;
a polymer encapsulating said active agent; and
a serum, wherein said serum is selected from the group consisting of whole untreated serum and a whole untreated serum in aqueous solution.

2. A microparticle composition according to claim 1, wherein said serum is selected from the group consisting of porcine serum, human serum and a serum autologous to an intended recipient of said microparticle.

3. A microparticle composition according to claim 1, wherein said polymer is a polyanhydride.

4. A microparticle composition according to claim 3, wherein said polyanhydride is poly(1,3-bis(p-carboxyphenoxy)propane-co-sebacic acid having a weight ratio of carboxyphenoxypropane to sebacic acid of between about 1:1 and about 1:8.

5. A microparticle composition according to claim 4, wherein said ratio of carboxyphenoxypropane to sebacic acid is about 1:4.

6. A microparticle composition according to claim 1 wherein said active agent is selected from the group consisting of an anti-proliferative agent, a matrix metalloproteinase inhibitor and an anti-cancer agent.

7. A microparticle composition of claim 6 wherein the anti-proliferative agent is rapamycin, paclitaxel or derivatives thereof.

8. A microparticle composition according to claim 1, wherein said microparticles have an average particle diameter of less than about 20 microns.

9. A microparticle composition according to claim 1, wherein said microparticles have an average particle diameter of about 1 micron or less.

10. A process for a preparing microparticle composition, comprising the steps of:
a) preparing an aqueous phase containing a serum, said serum being selected from a group consisting of whole untreated serum and whole, untreated serum in aqueous solution;
b) preparing an organic phase containing a polymer and an active agent;
c) mixing said aqueous phase and said organic phase to form an immiscible system; and
d) dispersing said two-phase system to form microparticles, said microparticles comprising said active agent and said polymer.

11. A process according to claim 10, wherein said serum is further selected from the group consisting of porcine serum, human serum and a serum autologous with an intended recipient of said microparticles.

12. A process according to claim 10, further comprising the step of redispersing said microparticles in serum.

13. A process according to claim 12, wherein said redispersing step comprises redispersing said microparticles in a serum having a concentration of at least 0.1%.

14. A process according to claim 10, wherein said serum has a concentration in said two-phase system of at least about 1.0% (v/v).

15. A process according to claim 10, wherein said polymer is a polyanhydride.

16. A process according to claim 10, wherein said polyanhydride is poly(1,3-bis(p-carboxyphenoxy)propane-co-sebacic acid.

17. A process according to claim 16, wherein said poly(1,3-bis(p-carboxyphenoxy)propane-co-sebacic acid has a weight ratio of carboxyphenoxypropane to sebacic acid of between about 1:1 and about 1:8.

18. A process according to claim 17, wherein said weight ratio is about 1:4.

19. A process according to claim 10, wherein said active agent is selected from the group consisting of an anti-proliferative agent, a matrix metalloproteinase inhibitor and an anti-cancer agent.

20. A microparticle composition prepared according to the process of claim 10.

21. A microparticle composition prepared according to the process of claim 10, wherein the anti-proliferative agent is rapamycin, paclitaxel or derivatives thereof.

22. A biodegradable composition comprising microparticles prepared according to claim 10 dispersed in a serum.

23. A biodegradable composition prepared according to the method of claim 22, wherein said serum is selected from the group consisting of whole untreated serum or whole untreated serum in aqueous solution.

24. A biodegradable composition according to claim 23, wherein said serum is further selected from the group consisting of porcine serum, human serum and a serum autologous with an intended recipient of said microparticles.

25. A biodegradable composition prepared according to the method of claim 22, wherein said microparticles have an average particle diameter of about 20 microns or less.

26. A biodegradable composition according to claim 25, wherein said microparticles have an average particle diameter of about 1 micron or less.

27. A method of treating a patient having a localized site of disease or injury, comprising the steps of:
a) providing a biodegradable composition, said composition including microparticles having an active agent; a polymer encapsulating said active agent and a serum associated with at least a portion of said polymer, and said serum being selected from the group consisting of whole untreated serum and whole untreated serum in aqueous solution; and
b) administering to said patient an effective amount of said composition.

28. A method according to claim 27, wherein said serum is selected from the group consisting of porcine serum, human serum and a serum that is autologous to an intended recipient of said serum.

29. A method according to claim 27 wherein said localized site is a vessel.

30. A method according to claim 29, wherein said vessel is a lymphatic vessel.

31. A method according to claim 25, wherein said localized site is within a coronary artery.

32. A method according to claim 25, wherein said localized site is myocardial tissue.

33. A method according to claim 25, wherein said localized site is a tumor.

34. A method according to claim 25 wherein said administering step is accomplished via a catheter.

33. A method according to claim 25 wherein said administering step is accomplished via an injection.

34. A method according to claim 25, herein said active agent is selected from the group consisting of an anti-proliferative agent, a matrix metalloproteinase inhibitor and an anti-cancer agent.

35. A method according to claim 25, wherein the anti-proliferative agent is rapamycin, paclitaxel or derivatives thereof.

36. A method according to claim 25, wherein said microparticles have an average particle diameter of about 20 microns or less.

37. A method according to claim 36, wherein said microparticles have an average particle diameter of about 1 micron or less.
